(19) United States
(12) Patent Application Publication
(10) Pub. No.: US 2025/0076325 A1
(43) Pub. Date: Mar. 6, 2025

(54) ARTIFICIAL HAPTEN AND ARTIFICIAL ANTIGEN OF FENTANYL, AND PREPARATION METHOD AND APPLICATION THEREOF

(71) Applicant: HANGZHOU ALLTEST BIOTECH CO., LTD., Hangzhou (CN)

(72) Inventors: Haijian WANG, Hangzhou (CN); Jian XU, Hangzhou (CN); Mengmeng XU, Hangzhou (CN); Zhaobin ZHAI, Hangzhou (CN); Lijuan KUANG, Hangzhou (CN)

(21) Appl. No.: 18/508,789

(22) Filed: Nov. 14, 2023

(30) Foreign Application Priority Data
Aug. 29, 2023 (CN) 202311096208.3

(51) Int. Cl. G01N 33/94; C07D 211/58

(52) U.S. Cl. CPC G01N 33/9486; C07D 211/58; G01N 2470/10

(57) ABSTRACT

Disclosed are an artificial hapten and artificial antigen of fentanyl, and a preparation method and application thereof. The artificial hapten of fentanyl according to the invention has an active group capable of being coupled with a carrier protein, and can be used as an antigenic determinant; and the artificial antigen of fentanyl further prepared can be immunized to obtain an anti-fentanyl antibody with high affinity, high sensitivity and strong specificity, and the antigen-antibody can be used in enzyme-linked immunity, immunochromatography and other technologies.

### CROSS REFERENCE TO RELATED APPLICATIONS

This application claims foreign priority of Chinese Patent Application No. 202311096208.3, filed on Aug. 29, 2023 in the China National Intellectual Property Administration, the disclosures of all of which are hereby incorporated by reference.

### TECHNICAL FIELD

The present invention belongs to the technical field of biochemical engineering, and particularly relates to an artificial hapten and artificial antigen of fentanyl, and a preparation method and application thereof.

### BACKGROUND OF THE PRESENT INVENTION

Fentanyl is a potent opioid analgesic, which is suitable for all kinds of pains, and analgesia after and during surgical and gynecological operations, is also used to prevent or alleviate postoperative delirium, may also be used in combination with an anesthetic as an auxiliary drug for anesthesia, and is used for large-scale dressing change and analgesia during a minor operation.

Existing data show that the harm of abuse of fentanyl firstly refers to the harm to human health. The fentanyl has a very strong pharmacological effect, which can directly affect a central nervous system and inhibit a respiratory center, leading to respiratory arrest and death. Secondly, the fentanyl is highly addictive. In the case of being addicted, not only a body suffers from the long-term harm, but also a social life is seriously affected, and the long-term abuse of fentanyl may cause the memory lapse and cognitive impairment of human beings, and may also lead to the heart function impairment, the physical and psychological dependence of human beings, and even inextricable addiction. Therefore, for a supervising purpose, it is necessary to develop a rapid detection method for fentanyl.

In the past, the fentanyl was mainly detected by high performance liquid chromatography (HPLC), gas chromatography (GC), thin layer chromatography (TLC), mass spectrometry (MS), and the like, but there were some shortcomings such as expensive instruments, time-consuming detection, and the need for professional technicians to operate, so that the requirements of modern detection for rapidity and accuracy could not be met. In recent years, with the development of immunoassay technology, the measurement of fentanyl by a colloidal gold method, an immunofluorescence method, an enzyme-linked immunoadsorption method, a chemiluminescence method, and the like has become a mainstream in a preliminary screening market at present.

The immunoassay technology can make up for all the above shortcomings, which is an analytical method for detecting various substances (drugs, hormones, proteins, microorganisms, and the like) by using an antigen-antibody specific binding reaction, and the key to establish immunoassay method for small molecular compounds is to produce antibodies with high affinity and strong specificity for the small molecular compounds. However, most small molecular compounds (with a molecular weight less than 1,000) comprising the fentanyl are not immunogenic, which means that the small molecular compounds lack T cell epitopes and cannot directly induce bodies of animals to generate specific antibodies, so that the small molecular substances are called haptens. By proper chemical modification, a hapten-carrier conjugate (which is namely an artificial antigen) is generated by attaching a connecting arm with an active group at an end portion on a certain position in a molecular structure of hapten, and then combining with a macromolecular carrier.

The artificial antigen may indirectly induce the proliferation and differentiation of B cells by means of the T cell epitopes, and then the specific antibodies are generated. Therefore, the synthesis of highly efficient artificial antigen is the premise and key to ensure immunoassay. Meanwhile, highly efficient artificial antigen and highly specific antibody may be used to prepare a colloidal gold immunochromatography detection reagent strip, which can meet the requirement of rapid on-site detection.

### SUMMARY OF THE PRESENT INVENTION

A first object of the present invention is to provide an artificial hapten of fentanyl aiming at the defects in the prior art, and the artificial hapten of fentanyl retains a characteristic structure of fentanyl to the greatest extent, and has an active group capable of being coupled with a carrier protein, so as to be used as an antigenic determinant.

The artificial hapten of fentanyl is provided, wherein a molecular structural formula of the artificial hapten of fentanyl is as shown in formula (I):

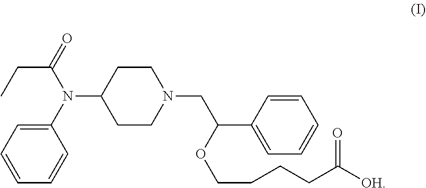 [SMILES: CCC(=O)N(c1ccccc1)C1CCN(CC(OCCCCC(=O)O)c2ccccc2)CC1]

A second object of the present invention is to provide a preparation method of the artificial hapten of fentanyl above, which comprises the following steps of:
step (1): dissolving β-hydroxyfentanyl (β-Hydroxy Fentanyl Hydrochloride) in N,N-dimethylformamide, adding sodium hydride, and stirring the mixture at room temperature for reaction for 0.5 hour; then adding ethyl 5-bromovalerate, and refluxing and stirring the mixture at 65° C. for reaction for 17 hours; and after ending the reaction, removing a solvent and extracting the mixture with ethyl acetate, and filtering, drying and purifying the mixture to obtain an oily substance A; wherein, a molar ratio of the β-hydroxyfentanyl to the sodium hydride is (1 to 1.5):3, and a molar ratio of the β-hydroxyfentanyl to the ethyl 5-bromovalerate is 1:(1.5 to 2);
under this reaction condition, the β-hydroxyfentanyl is easily dissolved in the N,N-dimethylformamide, a yield and a purity of the oily substance A are both high, and a post-treatment procedure is relatively simple;
preferably, the oily substance A is purified by thin-layer chromatography after filtering and drying, a volume ratio of the ethyl acetate to petroleum ether is 1:4, and an Rf value of the product is 0.2; and by-products or impurities in the reaction system may be removed by the thin-layer chromatography to achieve the purpose of approximate purification; and
step (2): dissolving the oily substance A with tetrahydrofuran and anhydrous methanol, adding 1 N to 1.5 N sodium hydroxide aqueous solution, quickly stirring the mixture at room temperature for reaction for 5 hours, adjusting a pH value to be 4 to 5 with 1 N hydrochloric acid solution, extracting the mixture with dichloromethane for multiple times, combining organic phases, drying and filtering the mixture, removing dichloromethane and then purifying the mixture to obtain the artificial hapten of fentanyl; wherein, a mass-volume ratio of the oily substance A to the tetrahydrofuran, the anhydrous methanol and the sodium hydroxide aqueous solution is 100 mg:(1.5 to 2) mL:(1.8 to 2) mL:10 mL.

Preferably, under this reaction condition, the oily substance A is easily hydrolyzed, has a relatively simple subsequent treatment procedure, and is easily purified.

Preferably, the target product may be extracted to the maximum extent by extracting with dichloromethane for three times.

Preferably, the purification is carried out by the thin-layer chromatography; the by-products or impurities in the reaction system may be removed by the thin-layer chromatography to achieve the purpose of approximate purification; and a volume ratio of 95 vol % ethanol to 1,4-dioxane, dichloromethane and 25 wt % ammonia water is 8:1:10:1, and an Rf value of the product is 0.6.

A reaction formula of the method is as shown in the following formula:

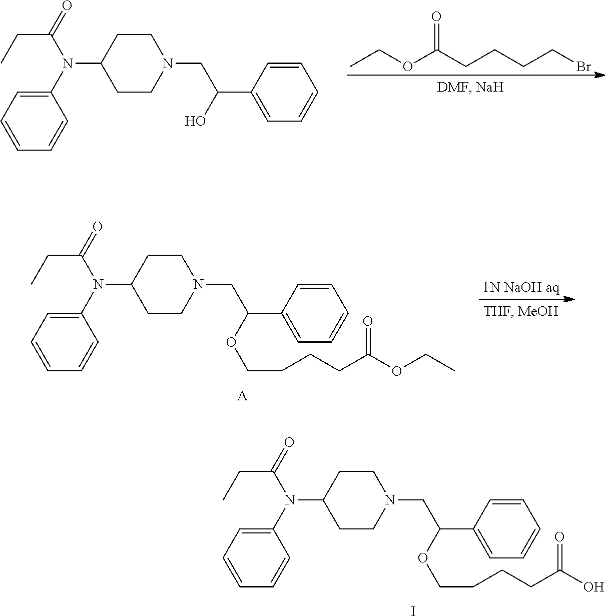

By the above method, a connecting arm is introduced onto hydroxyl of the β-hydroxyfentanyl, the introduction of the connecting arm at this modification site can retain a characteristic structure of fentanyl to the greatest extent, and this modification site is as far away from a characteristic functional group of fentanyl as possible, so as to expose a characteristic part to the maximum extent, thus avoiding an interference with a specific antigenic determinant, and being recognized by an immune body to the maximum extent.

Compared with a ring-shaped connecting arm, the connecting arm used in the present invention is chain-shaped, and a length of the connecting arm is appropriate, so that small molecules of fentanyl hapten can be fully exposed on a surface of an artificial antigen, and a recognition degree of T cells to the connecting arm during immunization is reduced as much as possible, thus making an antibody obtained by immunization have stronger specificity and affinity to fentanyl.

A third object of the present invention is to provide an artificial antigen of fentanyl obtained by coupling the artificial hapten of fentanyl above with a carrier protein, wherein a molecular structural formula of the artificial antigen of fentanyl is as shown in formula (II):

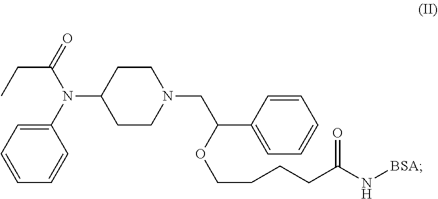

in formula (II), BSA refers to bovine serum albumin.

A fourth object of the present invention is to provide a preparation method of the artificial antigen of fentanyl above, which comprises: combining the artificial hapten of fentanyl with the bovine serum albumin by a mixed anhydride method to obtain the artificial antigen of fentanyl.

Specifically, the preparation of the artificial antigen of fentanyl by the mixed anhydride method comprises the following steps of:
step (1): dissolving the artificial hapten of fentanyl in a mixed solution of DMF and triethylamine for stirring in ice bath, adding isobutyl chloroformate during the stirring, and continuously stirring the mixture in ice bath for reaction for 2 hours to 2.5 hours; and after ending the reaction, centrifuging the mixture to obtain a supernatant; and
step (2): dropwise adding the supernatant obtained in the step (1) into a bovine serum albumin solution, standing the mixed solution obtained at 3° C. to 5° C. overnight, dialyzing and centrifuging the mixture, and taking a supernatant to obtain the artificial antigen of fentanyl.

Preferably, unless otherwise specified, the bovine serum albumin solution in the present invention is prepared by dissolving the bovine serum albumin in 0.01 M PBS buffer (with a pH value of 7.2 to 7.4).

Preferably, in the step (2), a concentration of the bovine serum albumin solution is 5 mg/mL, and a volume ratio of the supernatant to the bovine serum albumin solution is 1:5 to 6.

Compared with bovine gamma globulin (BGG), the bovine serum albumin (BSA) used as a macromolecular carrier in the present invention has the following advantages: \textcircled{1} the bovine serum albumin is the most commonly used carrier protein, which has higher immunogenicity and many antigenic determinants, and is easy to cause stronger immune response; \textcircled{2} molecules of the bovine serum albumin contain a large amount of lysine, have more free amino acids, and can maintain higher dissolvability under different PH values and ionic strengths, and when the bovine serum albumin is dissolved with an organic solvent (such as N,N-dimethylformamide), an active group of the bovine serum albumin is still in a dissolvable state, so that it is not easy to cause protein precipitation; and \textcircled{3} the bovine serum albumin has many lysine residues, contains hundreds of primary amines and carboxyl groups capable of becoming target spots cross-linking with cross-linking reagents such as glutaraldehyde, N-hydroxysuccinimide ester and EDC, is easy to couple with the hapten, with high coupling efficiency, and is not easy to produce a polyclonal antibody, so that it is easier to improve the specificity of antibody, and an anti-fentanyl antibody obtained by animal immunization of the artificial antigen of fentanyl formed by combining the bovine serum albumin with the artificial hapten of fentanyl has stronger specificity.

A fifth object of the present invention is to provide an anti-fentanyl antibody obtained by animal immunization of the artificial antigen of fentanyl, which may have a specific immune reaction with fentanyl.

Experiments show that a titer of immune serum obtained by immunizing New Zealand white rabbits with the artificial antigen of fentanyl is 1:85,000. It is indicated that the artificial antigen of fentanyl in the present invention can be immunized to obtain the anti-fentanyl antibody with high affinity, high sensitivity and strong specificity, and the anti-fentanyl antibody can be used in immunodetection and immunoassay of fentanyl.

A sixth object of the present invention is to provide an application of the anti-fentanyl antibody above in immunodetection of fentanyl in a sample.

Preferably, the sample comprises a body fluid sample, a tissue sample or an environmental sample. The body fluid sample refers to a mammal or human body fluid sample, which comprises urine, saliva, sweat, blood, and the like; the tissue sample refers to a mammal or human tissue sample, which comprises hair, nail, liver, and the like; and the environmental sample comprises powder, an object surface residue, a water sample, and the like.

Preferably, the immunodetection is an immunodetection or immunoassay method based on a principle of combining an antigen with an antibody, and comprises an enzyme-linked immunosorbent assay (ELISA), a competitive enzyme-linked immunosorbent assay or an immunochromatography assay.

Specifically, the anti-fentanyl antibody in the present invention may be used to prepare a competitive inhibition ELISA assay kit for fentanyl, which comprises the anti-fentanyl antibody, a fentanyl standard and an enzyme-labeled secondary antibody. Due to a limitation of a molecular structure of the fentanyl itself, the assay kit in the present invention is prepared by a principle of competitive inhibition ELISA. When in use, the fentanyl standard is coated first, a sample to be detected is added, and then the anti-fentanyl antibody and the enzyme-labeled secondary antibody are added in sequence for reaction, an OD450 value is detected, and a competitive inhibition rate is calculated. A coating amount of the fentanyl standard is appropriate to be a detection threshold of fentanyl by the anti-fentanyl antibody. The enzyme-labeled secondary antibody may be goat anti-mouse IgG-HRP.

Specifically, the anti-fentanyl antibody in the present invention may be used to prepare a competitive enzyme-linked immunosorbent assay kit, which comprises an enzyme-labeled plate wrapped with the anti-fentanyl antibody, a fentanyl standard, an enzyme-labeled antigen, a substrate and a termination solution. When in use, a sample to be detected and an enzyme-labeled antigen of fentanyl are jointly added into the enzyme-labeled plate for reaction, if fentanyl remains in the sample, the fentanyl may compete with the enzyme-labeled antigen of fentanyl to bind with the antibody on the plate, and then the substrate is added for reaction to terminate color development. The intensity of color development is inversely proportional to an amount of residual fentanyl in the sample. According to a standard curve made by the fentanyl standard, a content of fentanyl in the sample is calculated.

Specifically, the anti-fentanyl antibody in the present invention may be used to prepare a fentanyl immunodetection test strip, which comprises a sample pad, a marker pad, a reaction membrane and a sample suction pad; and the marker pad is wrapped with anti-fentanyl antibodies labeled with colored substances such as latex, colloidal gold or colored microspheres and labeled with colorless substances such as fluorescence, a detection line (T line) of the reaction membrane is wrapped with a fentanyl-bovine serum albumin complex, and a quality control line (C line) of the reaction membrane is wrapped with a secondary antibody. The test strip employs a principle of competition law, and a coating amount of the fentanyl-bovine serum albumin complex is also appropriate to be the detection threshold of fentanyl by the anti-fentanyl antibody. During detection, if only the C line develops color, it is indicated that the sample to be detected contains fentanyl and a content of fentanyl is detected to be higher than detection threshold; if the C line and the T line both develop color, it is indicated that the content of fentanyl in the sample to be detected is lower than the detection threshold, or there is no fentanyl in the sample to be detected; and if the C line and the T line do not develop color, it is indicated that the test strip has been invalid.

Compared with the prior art, the present invention has the beneficial effects as follows.

The modification site of the artificial hapten of fentanyl is novel in selection, and no precedent of artificial hapten with the same site is found at present. The artificial hapten of fentanyl in the present invention selects to retain the characteristic structure of fentanyl to the greatest extent, has the active group capable of being coupled with the carrier protein, and can be used as the antigenic determinant; and the artificial antigen of fentanyl further prepared can be immunized to obtain the anti-fentanyl antibody with high affinity, high sensitivity and strong specificity, the titer of immune serum obtained by immunizing New Zealand white rabbits is 1:85,000, and the artificial antigen of fentanyl may be used for rapid and accurate immunodetection and immunoassay of fentanyl.

### DESCRIPTION OF THE DRAWINGS

FIG. 1 is a flow chart of preparation of an artificial antigen II of fentanyl in the present invention;

wherein, DMF represents N,N-dimethylformamide, NaH represents sodium hydride, THF represents tetrahydrofuran, MeOH represents anhydrous methanol, RT represents room temperature, Et₃N represents triethylamine, and BSA represents bovine serum albumin, the same below;

FIG. 2 is a liquid chromatogram of an artificial hapten I of fentanyl in the present invention;

wherein, an abscissa of the liquid chromatogram is time, in a unit of min; and an ordinate of the liquid chromatogram is a response value, in a unit of mAU;

FIG. 3 is a mass spectrogram of the artificial hapten I of fentanyl in the present invention;

wherein, Relative Abundance represents a relative abundance; and m/z represents a charge-mass ratio;

FIG. 4 is an ultraviolet scanning image of the artificial hapten I of fentanyl, the artificial antigen II of fentanyl and bovine serum albumin;

wherein, Abs represents an ultraviolet-visible absorption spectrum, and WL (nm) represents a wavelength (nm);

FIG. 5 is a flow chart of preparation of an artificial antigen IV of fentanyl in Comparative Example 1;

wherein, Pyridine represents pyridine, and DCC represents dicyclohexylcarbodiimide, the same below;

FIG. 6 is a flow chart of preparation of an artificial antigen V of fentanyl in Comparative Example 2;

wherein, BGG represents bovine gamma globulin, the same below;

FIG. 7 is a flow chart of preparation of an artificial antigen VI of fentanyl in Comparative Example 3;

FIG. 8 is a flow chart of preparation of an artificial antigen VII of fentanyl in Comparative Example 4;

FIG. 9 is a flow chart of preparation of an artificial antigen VIII of fentanyl in Comparative Example 5;

FIG. 10 is a flow chart of preparation of an artificial antigen IX of fentanyl in Comparative Example 6; and

FIG. 11 is a flow chart of preparation of an artificial antigen X of fentanyl in Comparative Example 7.

### DETAILED DESCRIPTION OF PREFERRED EMBODIMENTS

The present invention is further described in detail hereinafter with reference to the drawings and specific embodiments.

### Embodiment 1

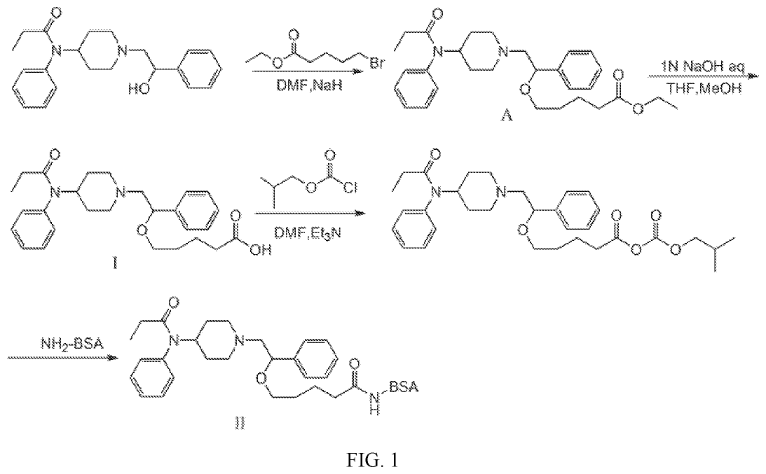

This embodiment provided a preparation method of an artificial antigen II of fentanyl (with a reaction mechanism as shown in FIG. 1), which comprised the following steps.

### (1) Preparation of Artificial Hapten I:

\textcircled{1} 181 mg (0.514 mmol) of β-hydroxyfentanyl was dissolved with 5 ml of N,N-dimethylformamide, placed in a 50 ml round-bottomed flask, added with 37 mg (1.542 mmol) of sodium hydride, stirred at room temperature for reaction for 0.5 hour, added with 122 μL (0.771 mmol) of ethyl 5-bromovalerate, and refluxed and stirred at 65° C. for reaction for 17 hours. After ending the reaction, the mixture was directly rotationally dried to obtain 298 mg of yellow oily substance, and added with 20 ml×2 ethyl acetate to extract twice. Organic phases were collected, directly rotationally dried to obtain 268 mg of yellow oily substance, and purified by thin-layer chromatography to obtain 196 mg of light yellow oily substance A. A solvent and an eluent were anhydrous ethanol.

The light yellow oily substance A was subjected to TLC detection, in which a chromatographic solution was ethyl acetate:petroleum ether=4:1 (volume ratio), and an Rf value of the product was 0.2.

A chromatographic solution used in the thin-layer chromatography was ethyl acetate:petroleum ether=4:1 (volume ratio), and an Rf value of the product was 0.2.

\textcircled{2} 196 mg (0.408 mmol) of light yellow oily substance A was dissolved with 2.94 ml of tetrahydrofuran and 3.53 ml of anhydrous methanol, and added with 19.6 ml of 1 N sodium hydroxide aqueous solution, and the solution become turbid. The mixture was quickly stirred at room temperature for reaction for 5 hours, a pH value was adjusted to be 4 to 5 with 1 N hydrochloric acid solution, and the mixture was extracted with 30 ml×3 dichloromethane for three times. Organic phases were combined, dried, filtered, rotationally dried, and purified by thin-layer chromatography to obtain 170 mg of artificial hapten I of fentanyl. A solvent and an eluent were anhydrous ethanol.

The artificial hapten of fentanyl was subjected to TLC detection, in which a chromatographic solution was 95 vol % ethanol:1,4-dioxane:dichloromethane:25 wt % ammonia water=8:1:10:1, and an Rf value of the product was 0.6.

A chromatographic solution used in the thin-layer chromatography was 95 vol % ethanol:1, 4-dioxane:dichloromethane:25 wt % ammonia water=8:1:10:1, a solvent and an eluent were anhydrous ethanol, and an Rf value of the product was 0.6.

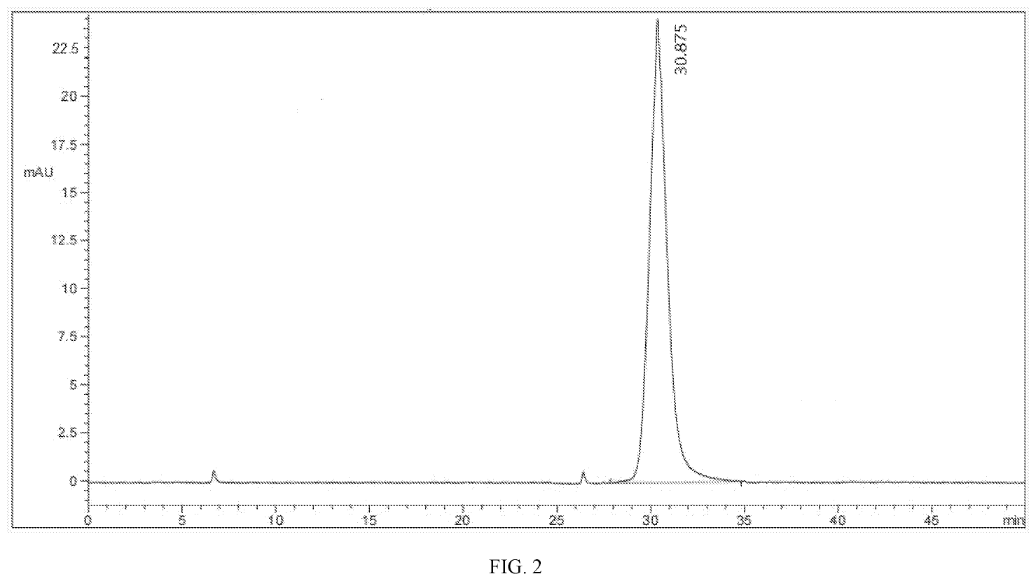

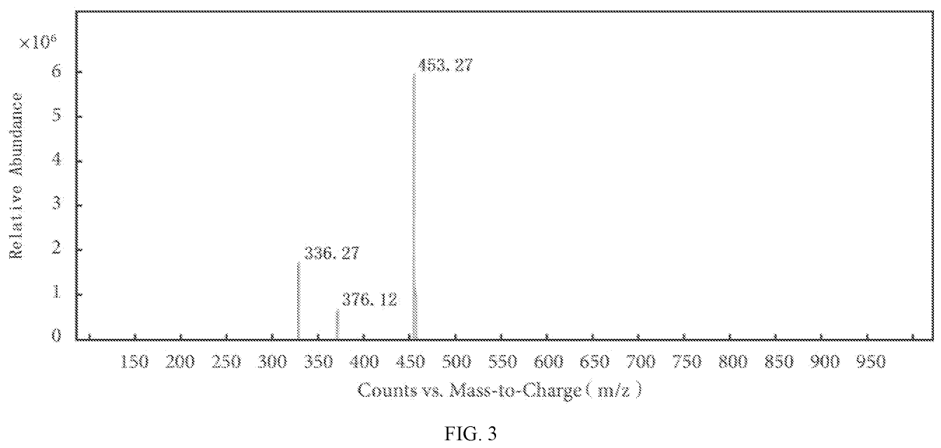

A liquid chromatogram of the artificial hapten I of fentanyl was shown in FIG. 2 (ultraviolet detector, wavelength 288 nm), and a mass spectrogram of the artificial hapten I of fentanyl was shown in FIG. 3.

It can be seen from FIG. 2 that a purity of the artificial hapten of fentanyl obtained by purification reaches more than 99.9%. It can be seen from FIG. 3 that a mass-to-charge ratio (m/z) of an M+H peak of the artificial hapten of fentanyl obtained in this embodiment is 453.27, which is consistent with a theoretical relative molecular weight of 452, and mass-to-charge ratios (m/z) of ion peaks of other two main fragments are 336.27 and 376.12 respectively, which are consistent with theoretical molecular weights of 335 and 375 of other two main fragments. Based on the above data, it can be preliminarily determined that the final compound obtained in the step \textcircled{2} is the artificial hapten I of fentanyl designed in the present invention.

### (2) Preparation of Artificial Antigen II of Fentanyl:

\textcircled{3} 170 mg (0.376 mmol) of the artificial hapten I of fentanyl was placed in a 50 ml round-bottomed flask, added with 8.5 ml of N,N-dimethylformamide (DMF), then added with 52 μL (0.376 mmol) of triethylamine, stirred in an ice bath for 30 minutes, then added with 97 μL (0.752 mmol) of isobutyl chloroformate, stirred in an ice bath for 2 hours, and centrifuged after ending the reaction, and a supernatant was taken for later use.
\textcircled{4} 14.5 g (0.0405 mol) of disodium hydrogen phosphate dodecahydrate, 43.875 g (0. 75 mol) of sodium chloride and 1.495 g (0.00958 mol) of sodium dihydrogen phosphate dihydrate were weighed and dissolved in double distilled water to a constant volume of 5.0 L to obtain 0.01 M PBS buffer with a pH value of 7.4.

\textcircled{5} 0.215 g of bovine serum albumin was weighed and dissolved in 43 ml of PBS buffer in the step \textcircled{4} to obtain a bovine serum albumin solution with a concentration of 5 mg/ml.
\textcircled{6} Under rapid stirring, the supernatant in the step \textcircled{3} was slowly dropwise added into the bovine serum albumin solution, a volume ratio of the supernatant to the bovine serum albumin solution was 1:5, and the obtained mixed solution was kept standing at 4° C. overnight to obtain a mixed solution of artificial antigen.

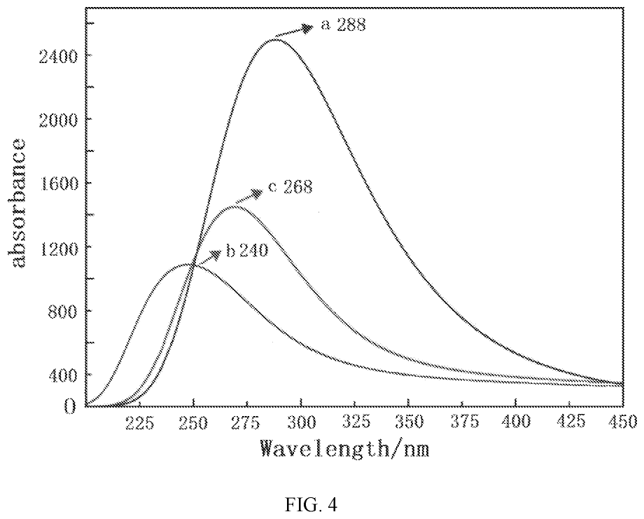

\textcircled{7} The mixed solution of artificial antigen was transferred into a dialysis bag, dialyzed with the PBS buffer in the step \textcircled{4} for nine times, and centrifuged after ending the dialysis, and a supernatant was taken to obtain an artificial antigen II of fentanyl-bovine serum albumin conjugate. An ultraviolet scanning image of the artificial antigen II of fentanyl before and after preparation was shown in FIG. 4.

In FIG. 4, a curve a is an ultraviolet scanning image of the artificial hapten I of fentanyl, a curve b is an ultraviolet scanning image of the artificial antigen II of fentanyl, and a curve c is an ultraviolet scanning image of the bovine serum albumin. A maximum absorption wavelength of the artificial hapten I of fentanyl is 288 nm, a maximum absorption wavelength of the bovine serum albumin is 268 nm, and a maximum absorption wavelength of the artificial antigen II of fentanyl is 240 nm. Compared with the artificial hapten I of fentanyl and the bovine serum albumin, the maximum absorption wavelength of the artificial antigen II of fentanyl is changed obviously, which indicates that the artificial hapten I of fentanyl is successfully coupled with the bovine serum albumin.

### Comparative Example 1

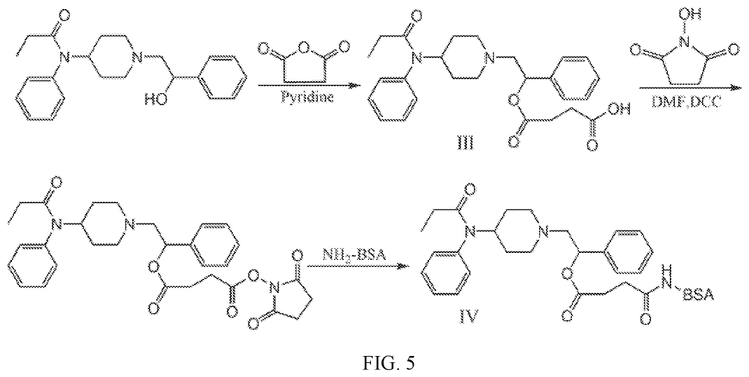

This embodiment provided a preparation method of an artificial antigen IV of fentanyl (with a reaction mechanism as shown in FIG. 5), which comprised the following steps.

### (1) Preparation of Artificial Hapten III of Fentanyl:

\textcircled{1} 181 mg (0.514 mmol) of β-hydroxyfentanyl was dissolved with 20 ml of pyridine, placed in a 50 ml single-mouth round-bottomed flask, added with 103 mg (1.03 mmol) of butanedioic anhydride, and refluxed and stirred at 100° C. for reaction for 17 hours. After ending the reaction, the mixture was directly rotationally dried to obtain 256 mg of yellow oily substance, and purified by thin-layer chromatography to obtain 180 mg of the artificial hapten III of fentanyl. A solvent and an eluent were anhydrous ethanol.

The artificial hapten III of fentanyl was subjected to TLC detection, in which a chromatographic solution was 95 vol % ethanol:1,4-dioxane:dichloromethane:25 wt % ammonia water=8:1:10:1 (volume ratio), and an Rf value of the product was 0.5.

A chromatographic solution used in the thin-layer chromatography was 95 vol % ethanol:1,4-dioxane:dichloromethane:25 wt % ammonia water=8:1:10:1 (volume ratio), and an Rf value of the product was 0.5.

### (2) Preparation of Artificial Antigen IV of Fentanyl:

\textcircled{2} 180 mg (0.398 mmol) of the artificial hapten III of fentanyl was placed in a 50 ml round-bottomed flask, added with 9 ml of N,N-dimethylformamide (DMF), then added with 69 mg (0.6 mmol) of N-hydroxysuccinimide (NHS) and 124 mg (0.602 mmol) of dicyclohexylcarbodiimide (DCC), stirred at room temperature for reaction overnight, and centrifuged after ending the reaction, and a supernatant was taken for later use.

\textcircled{3} 14.5 g (0.0405 mol) of disodium hydrogen phosphate dodecahydrate, 43.875 g (0.75 mol) of sodium chloride and 1.495 g (0.00958 mol) of sodium dihydrogen phosphate dihydrate were weighed and dissolved in double distilled water to a constant volume of 5.0 L to obtain 0.01 M PBS buffer with a pH value of 7.4.
\textcircled{4} 0.225 g of bovine serum albumin was weighed and dissolved in 45 ml of PBS buffer in the step 33 to obtain a bovine serum albumin solution with a concentration of 5 mg/ml.

\textcircled{5} Under rapid stirring, the supernatant in the step (was slowly dropwise added into the bovine serum albumin solution, a volume ratio of the supernatant to the bovine serum albumin solution was 1:5, and the obtained mixed solution was kept standing at 4° C. overnight to obtain a mixed solution of artificial antigen.

The mixed solution of artificial antigen was transferred into a dialysis bag, dialyzed with the PBS buffer in the step 33 for nine times, and centrifuged after ending the dialysis, and a supernatant was taken to obtain an artificial antigen IV of fentanyl-bovine serum albumin conjugate.

### Comparative Example 2

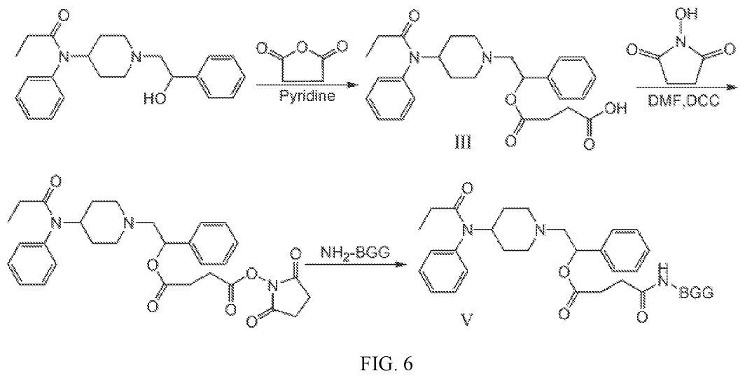

This embodiment provided a preparation method of an artificial antigen V of fentanyl (with a reaction mechanism as shown in FIG. 6), which comprised the following steps.

### (1) Preparation of Artificial Hapten III of Fentanyl:

\textcircled{1} was the same as that in Comparative Example 1.

### (2) Preparation of Artificial Antigen V of Fentanyl:

Bovine gamma globulin was used as a carrier to couple with the artificial hapten III of fentanyl, and the coupling steps \textcircled{2} to \textcircled{6} were the same as those in Comparative Example 1, so that the artificial antigen V of fentanyl was obtained.

### Comparative Example 3

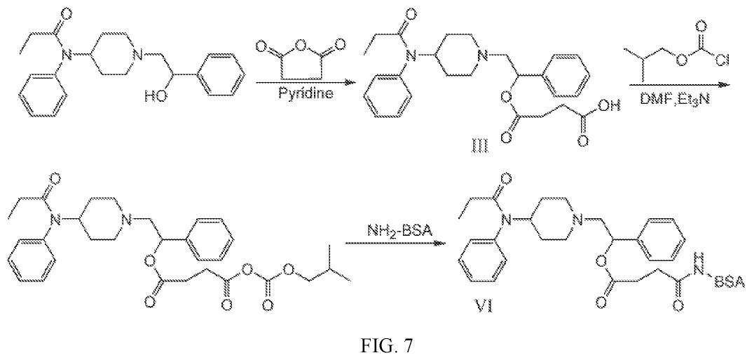

This embodiment provided a preparation method of an artificial antigen VI of fentanyl (with a reaction mechanism as shown in FIG. 7), which comprised the following steps.

### (1) Preparation of Artificial Hapten III of Fentanyl:

\textcircled{1} was the same as that in Comparative Example 1.

(2) Preparation of Artificial Antigen VI of Fentanyl:

\textcircled{2} 180 mg (0.398 mmol) of the artificial hapten III of fentanyl was placed in a 50 ml round-bottomed flask, added with 9 ml of N,N-dimethylformamide (DMF), then added with 55 μL (0.398 mmol) of triethylamine, stirred in an ice bath for 30 minutes, then added with 103 μL (0.796 mmol) of isobutyl chloroformate, continuously stirred in an ice bath for 2 hours, and centrifuged after ending the reaction, and a supernatant was taken for later use.

\textcircled{3} to \textcircled{6} were the same as those in Comparative Example 1, so that the artificial antigen VI of fentanyl was obtained.

### Comparative Example 4

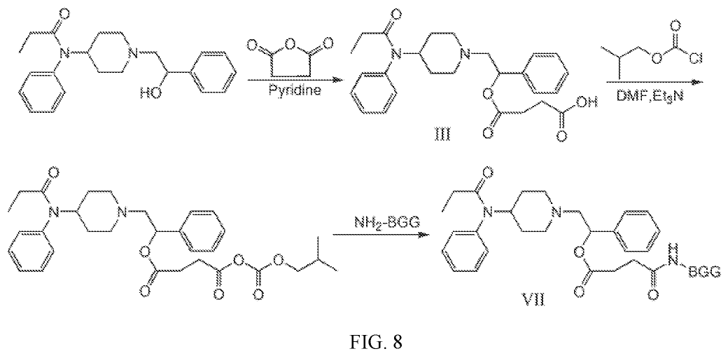

This embodiment provided a preparation method of an artificial antigen VII of fentanyl (with a reaction mechanism as shown in FIG. 8), which comprised the following steps.

### (1) Preparation of Artificial Hapten III of Fentanyl:

\textcircled{1} was the same as that in Comparative Example 1.

### (2) Preparation of Artificial Antigen of Fentanyl:

Bovine gamma globulin was used as a carrier to couple with the artificial hapten III of fentanyl, and the coupling steps \textcircled{2} to \textcircled{6} were the same as those in Comparative Example 3, so that the artificial antigen VII of fentanyl was obtained.

### Comparative Example 5

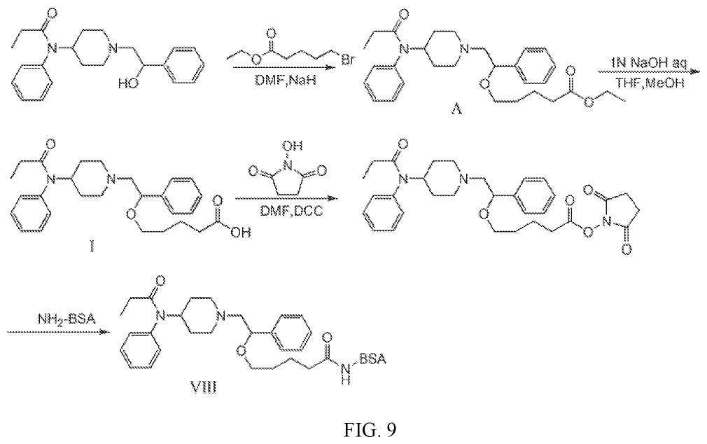

This embodiment provided a preparation method of an artificial antigen VIII of fentanyl (with a reaction mechanism as shown in FIG. 9), which comprised the following steps.

### (1) Preparation of Artificial Hapten I of Fentanyl:

\textcircled{1} to \textcircled{2} were the same as those in Embodiment 1.

### (2) Preparation of Artificial Antigen VIII of Fentanyl:

\textcircled{3} 170 mg (0.376 mmol) of the artificial hapten I of fentanyl was placed in a 50 ml round-bottomed flask, added with 8.5 ml of N, N-dimethylformamide (DMF), then added with 65 mg (0.564 mmol) of N-hydroxysuccinimide (NHS) and 116 mg (0.564 mmol) of dicyclohexylcarbodiimide (DCC), stirred at room temperature for reaction overnight, and centrifuged after ending the reaction, and a supernatant was taken for later use.

\textcircled{4} to \textcircled{7} were the same as those in Embodiment 1, so that the artificial antigen VIII of fentanyl was obtained.

### Comparative Example 6

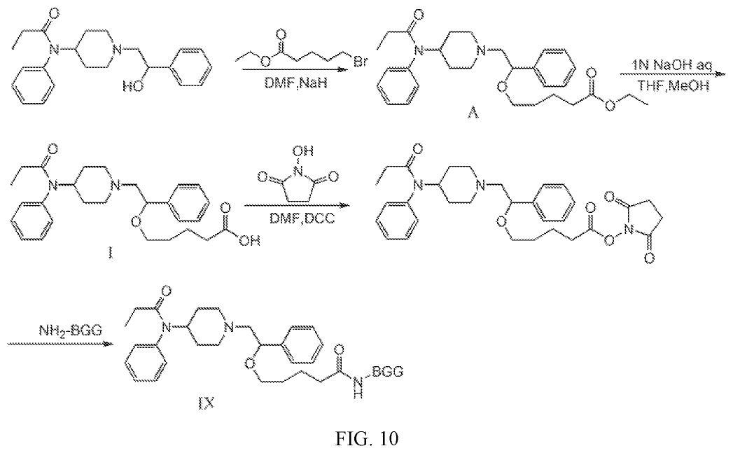

This embodiment provided a preparation method of an artificial antigen IX of fentanyl (with a reaction mechanism as shown in FIG. 10), which comprised the following steps.

### (1) Preparation of Artificial Hapten I of Fentanyl:

\textcircled{1} to \textcircled{2} were the same as those in Embodiment 1.

(2) Preparation of artificial antigen IX of fentanyl:

Bovine gamma globulin was used as a carrier to couple with the artificial hapten I of fentanyl, and the coupling steps \textcircled{3} to \textcircled{7} were the same as those in Comparative Example 5, so that the artificial antigen IX of fentanyl was obtained.

### Comparative Example 7

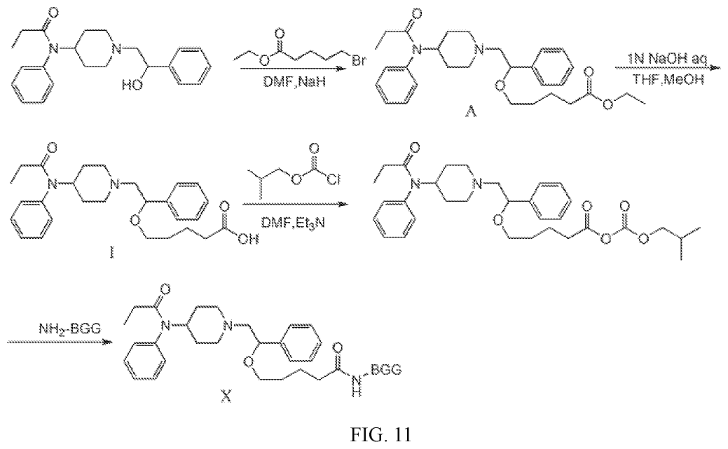

This embodiment provided a preparation method of an artificial antigen X of fentanyl (with a reaction mechanism as shown in FIG. 11), which comprised the following steps.

### (1) Preparation of Artificial Hapten I of Gentanyl:

\textcircled{1} to \textcircled{2} were the same as those in Embodiment 1.

### (2) Preparation of Artificial Antigen X of Gentanyl:

Bovine gamma globulin was used as a carrier to couple with the artificial hapten I of fentanyl, and the coupling steps 3 to (were the same as those in Embodiment 1, so that the artificial antigen X of fentanyl was obtained.

### Application Example 1

### Colloidal Gold Immunochromatography Detection Reagent Strip of Fentanyl

### (1) Preparation of Colloidal Gold

Colloidal gold particles were prepared by a trisodium citrate reduction method with a simple preparation procedure, and a conical flask filled with 100 ml of 0.01% HAuCl₄ solution was placed on a magnetic stirrer, stirred and heated to boiling, and quickly added with a certain amount of 1% trisodium citrate. Meanwhile, a stirring speed was adjusted, the mixture showed some blue color at first and then showed a light blue color and a blue color, and then was heated to show a red color, the heating was stopped when the mixture showed a transparent orange red color after boiling for 7 minutes to 10 minutes, and the heated mixture was kept for later use.

### (2) Colloidal Gold-Labeled Fentanyl Antibody

10 ml of colloidal gold solution was added into a 50 ml centrifuge tube, and stirred evenly, and a pH value was adjusted to be an optimal pH value with 0.1 M K₂CO₃. An appropriate amount of (mouse IgG) FYL-Ab was gradually dropwise added during slow stirring, and then evenly mixed and stood for reaction for 30 minutes. Subsequently, the mixture was slowly stirred continuously, slowly dropwise added with PEG 20,000 (with a final concentration of 0.05%), and evenly mixed and stood for 30 minutes. The mixture was centrifuged at 4° C. under 8,000 rpm for 30 minutes. After centrifugation, a supernatant was carefully absorbed, redissolved with 15 mM Tris buffer containing 0.05% PEG 20,000, and evenly mixed and stood for reaction for 30 minutes. 0.05% sodium azide was added for anticorrosion; and the mixture was repeatedly centrifuged at 4° C. under 8,000 rpm for 30 minutes. A supernatant of the mixture was discarded by centrifugation, an OD value of the mixture was measured by an ultraviolet spectrophotometer, and then the mixture was kept for later use.

### (3) Treatment of Polyester Fiber Membrane

| Reagent | Dosage |
|---|---|
| Purified water | 90% |
| Na₂HPO₄•12H₂O | 5.74 g/L |
| K₂HPO₄ | 1.46 g/L |
| Tween-20 | 0.1% |

A treatment solution of polyester fiber membrane was prepared according to the above formula, and finally, purified water was used to reach a constant volume. A pH value was adjusted to be 7.4+0.1 with 6 M HCL and 6 M NaOH. 84 mm×301 mm polyester membrane was rolled up and placed into a 50 ml centrifuge tube, added with an appropriate amount of the solution prepared above, placed in a rotary mixer and mixed at a uniform speed for 5 hours, then taken out and laid flat on a grid rack, and dried at 37° C.

### (4) Treatment of Sample Pad

| Reagent | Dosage |
|---|---|
| Purified water | 90% |
| Borax | 19.05 g/L |
| S-17 | 5 g/L |
| Sodium cholate | 5 g/L |
| PVP polyvinylpyrrolidone | 10 g/L |
| Sodium azide | 0.02% |

A treatment solution of glass fiber membrane was prepared according to the above formula, and finally, purified water was used to reach a constant volume. A pH value was adjusted to be 8.5±0.1 with 6 M HCL and 6 M NaOH, untreated glass fiber membrane was taken out, laid flat on a grid rack, and evenly sprayed with the solution prepared above by a disposable syringe, the solution was evenly coated by a cover end of 50 ml disposable centrifugal tube, and the grid rack was placed into an oven at 37° C. for drying.

### (5) Assembly of Immunochromatography Detection Reagent Strip

A sample pad, a conjugate pad (with dry colloidal gold-labeled mouse IgG and FYL-Ab on the polyester fiber membrane), a nitrocellulose membrane (wrapped with goat anti-mouse IgG and FYL-Ag) and a water absorbent pad were sequentially adhered to a plastic plate from bottom to top. Various joint parts were overlapped by about 1.5 mm. When a detection sample was dropwise added to a sample position, the sample was chromatographed upwardly by using a capillary effect to complete the reaction.

### (6) Interpretation of Results

Positive result: one red line appeared on the C line (quality control line) in the reagent strip display area; negative result: one red line appeared on the C line (quality control line) and one red line appeared on the T line (detection line) in the reagent strip display area; invalid result: no red line appeared on the C line (quality control line) and no red line appeared on the T line (detection line) in the reagent strip display area, or only one red line appeared on the T line. The above application example is not intended to restrict the present invention, the present invention is not merely limited to the above application example, and so long as it meets the requirements of the present invention, it belongs to the scope of protection of the present invention.

### Detection Example 1

### Performance Measurement of Artificial Antigen of Fentanyl

### (1) Identification of Artificial Antigen of Fentanyl:

Molar absorption coefficient ε: artificial hapten solutions of fentanyl with concentrations of 0 μg/ml, 5 μg/ml, 10 μg/ml, 20 μg/ml, 30 μg/ml and 40 μg/ml were prepared with a PBS buffer, an ultraviolet scanning image showed that a maximum absorption wavelength of the artificial hapten of fentanyl was 240 nm, an absorbance was measured at 240 nm, and a parallel sample was made for each concentration. A calculation formula of molar light absorption coefficient (molar absorption coefficient) was: ε=absorbance/molar concentration.

Measurement of protein concentration of conjugate: 1 ml of bovine serum albumin solutions with concentrations of 0 μg/ml, 10 μg/ml, 20 μg/ml, 30 μg/ml, 40 μg/ml, 60 μg/ml, 80 μg/ml, 100 μg/ml and 120 μg/ml were respectively prepared with a PBS buffer, added with 3 ml of Coomassie brilliant blue staining solution, mixed immediately, and warmed in a water bath at 30° C. for 5 minutes, a parallel sample was made for each concentration, an absorbance was measured at 655 nm, and a relationship curve between the protein concentration and the absorbance was drawn. The artificial antigen solution of fentanyl (prepared with the PBS buffer) was diluted in a certain proportion, an absorbance of the artificial antigen of fentanyl was measured at 655 nm, and a corresponding protein concentration value of the artificial antigen solution of fentanyl was read from the curve.

Measurement of coupling ratio: 100 μg/ml bovine serum albumin PBS solution was prepared, a conjugate (artificial antigen of fentanyl) was diluted to 100 μg/ml with PBS, an absorbance A, was measured at 240 nm, and an absorbance A2 was measured with PBS as blank, so that the coupling ratio γ was: γ=[(A₁-A₂)/ε]/(100×10⁻³/66,000),

wherein ε was the molar light absorption coefficient (L/mol), 66,000 was a molecular weight of bovine serum albumin, and 100×10³ was a concentration of bovine serum albumin (g/L).

When the bovine serum albumin was used as a carrier, a calculation formula of the coupling ratio was: γ=[(A₁−A₂)/ε]/(100×10⁻³/43,000), wherein 43,000 was a molecular weight of bovine gamma globulin.

TABLE 1
Coupling ratios and molar absorption coefficients of
various artificial antigens of fentanyl

| | Arti- | Cou- | Protein | Molar |
|---|---|---|---|---|
| | ficial | pling | concentration | absorption |
| Serial number | antigen | ratio | of conjugate | coefficient |
| Embodiment 1 | II | 28 | 3.568 mg/ml | 5928.88 |
| Comparative Example 1 | IV | 20 | 3.124 mg/ml | 6018.38 |
| Comparative Example 2 | V | 10 | 1.685 mg/ml | 6018.38 |
| Comparative Example 3 | VI | 16 | 2.687 mg/ml | 6018.38 |
| Comparative Example 4 | VII | 6 | 0.354 mg/ml | 6018.38 |
| Comparative Example 5 | VII | 24 | 3.356 mg/ml | 5928.88 |
| Comparative Example 6 | IX | 8 | 0.896 mg/ml | 5928.88 |
| Comparative Example 7 | X | 12 | 2.088 mg/ml | 5928.88 |

It can be seen from Table 1 that the structure of the artificial hapten, the activation method of the artificial hapten and the structure of the carrier protein all have effects on a binding ratio of the artificial hapten and the carrier protein during crosslinking.

### (2) Animal Immunization

The various artificial antigens of fentanyl prepared were used to immunize New Zealand white rabbits, and titers of obtained immune serums were detected by ELISA. Detection results were shown in Table 2.

TABLE 2
Detection results of titers of various immune serums

| | Artificial antigen of | Titer of immune |
|---|---|---|
| Serial number | fentanyl | serum |
| Embodiment 1 | II | 1:85000 |
| Comparative Example | IV | 1:12000 |
| 1 | | |
| Comparative Example | V | 1:6400 |
| 2 | | |
| Comparative Example | VI | 1:18000 |
| 3 | | |
| Comparative Example | VII | / |
| 4 | | |
| Comparative Example | VIII | 1:18000 |
| 5 | | |
| Comparative Example | IX | / |
| 6 | | |
| Comparative Example | X | 1:14000 |
| 7 | | |

It can be seen from Table 2 that, compared with Embodiment 1, the titers of the immune serums obtained by animal immunization with the artificial antigens of fentanyl in various comparative examples are all relatively low, thus being unable to be used in immunoassay. The artificial antigens VII and IX of fentanyl obtained in Comparative Example 4 and Comparative Example 6 immediately produce a large number of precipitates during dialysis. The artificial antigen V of fentanyl obtained in Comparative Example 2 is turbid and produces a large number of precipitates after frozen storage, with very poor stability, thus being unable to be used as an immune antigen. However, the titer of the immune serum obtained by animal immunization with the artificial antigen II of fentanyl reaches 1:85,000, thus being able to be completely used in immunoassay, so that a more convenient, rapid and accurate way is provided for the detection of fentanyl.

### Detection Example 2

### Performance Measurement of Colloidal Gold Immunochromatography Detection Reagent Strip of Fentanyl Urine

In this detection example, a functional test was carried out on the colloidal gold immunochromatography detection reagent strip of fentanyl prepared in Application Example 1, and specific operations were as follows.

### (1) Sample Preparation

Fresh clinically negative urine was collected, and added with a fentanyl standard to prepare samples with different concentrations: 0 ng/ml, 0.25 ng/ml, 0.5 ng/ml, 0.75 ng/ml, 1 ng/ml, 1.25 ng/ml, 1.5 ng/ml, 1.75 ng/ml, 2 ng/ml and 3 ng/ml.

Cross-interfering substance solution: the negative urine was added with different poisons and common drugs to prepare solution samples with a concentration of 100 μg/ml.

### (2) Detection and Result Analysis

The above samples were dropwise added to a sample loading area of a reagent card, and when there was color development on both the detection line and the quality control line of the reagent strip, the detection result was negative (−); when there was color development on the quality control line but there was no color development on the detection line, the detection result was positive (+); and when there was no color development on the quality control line but there was color development on the detection line, or there was no color development on both the quality control line and the detection line, the detection result was invalid (X).

Sensitivity experiment results in this detection example were shown in Table 3, a minimum detection limit of the colloidal gold immunochromatography reagent strip of fentanyl urine was 1 ng/ml, detection results higher than 1 ng/ml were all positive, and detection results lower than 1 ng/ml were all negative.

A drug cross-interference experiment was shown in Table 4, 88 common drugs were prepared into samples with a concentration of 100 μg/ml with the negative urine, and results were all negative.

TABLE 3
Sensitivity experiment results of colloidal gold
immunochromatography reagent strip of fentanyl urine

| Concentration | |
|---|---|
| (μg/ml) | Result |
| 0 | −, −, −, −, − |
| 0.25 | −, −, −, −, − |
| 0.5 | 7, 7, −, −, − |
| 0.75 | −, −, −, −, − |
| 1 | −, +, +, −, + |
| 1.25 | +, +, +, +, + |
| 1.5 | +, +, +, +, + |
| 1.75 | +, +, +, +, + |
| 2 | +, +, +, +, + |
| 3 | +, +, +, +, + |

TABLE 4
Drug cross-interference experiment results of colloidal gold immunochromatography reagent strip of fentanyl

| Reagent | Result | Reagent | Result | Reagent | Result | Reagent | Result |
|---|---|---|---|---|---|---|---|
| Acetaminophen | − | Nifedipine | − | Oxolinic acid | − | Propranolol | − |
| Cyclobenzaprine | − | Ampicillin | − | Tryptophan | − | Prednisone | − |
| Methoxyphenamine | − | Erythromycin | − | Bilirubin | − | Lidocaine | − |
| Sulindac | − | Norethindrone | − | Tyrosine | − | Pseudo ephedrine | − |
| Phenacetin | − | Venlafaxine | − | Papaverine | − | 5-hydroxytryptamine | − |
| | | hydrochloride | | hydrochloride | | | |
| Desipramine | − | Nortriptyline | − | Bupropion | − | Clomipramine | − |
| | | | | hydrochloride | | | |
| Acecainide | − | Verapamil | − | Ibuprofen | − | Loperamide | − |
| Vitamin B1 | − | Vitamin C | − | Penicillin | − | Quinine | − |
| Thioridazine | − | Fenoprofen | − | Caffeine | − | Sulfadimidine | − |
| Nalidixic acid | − | Narcotine | − | Trimipramine | − | Clonidine | − |
| Triamterene | − | Aspartame | − | Perphenazine | − | Maprotiline | − |
| | | | | | | hydrochloride | |
| Salbutamol | − | Furosemide | − | Carbamazepine | − | Ranitidine | − |
| Naloxone | − | Gentisic acid | − | Phencyclidine | − | Tetrahydrocortisone | − |
| Trifluoperazine | − | Octopamine | − | Hydroxytyramine | − | Hydrocortisone | − |
| | | | | | | acetate | |
| Digoxin | − | Benzilic acid | − | Chloramphenicol | − | Tyramine | − |
| Naltrexone | − | Ecgonidine | − | Ketamine | − | Levo-cotinine | − |
| | | methyl ester | | | | | |
| Trimethoprim | − | Benzoic acid | − | Phenobarbital | − | Meprobamate | − |
| Amitriptyline | − | Bovine | − | Nicotinamide | − | Salicylic acid | − |
| | | hemoglobin | | | | | |
| Diphenhydramine | − | Oxazepam | − | Chlorothiazide | − | Creatinine | − |
| hydrochloride | | | | | | | |
| Naproxen | − | Chloral hydrate | − | Ketoprofen | − | Methaqualone | − |
| Doxepin | − | Benzoylecgonine | − | Chlorpromazine | − | Secobarbital | − |
| Amobarbital | − | Hydralazine | − | Labetalol | − | Methapyrilene | − |

We claim:

1. An artificial hapten of fentanyl, wherein a molecular structural formula of the artificial hapten of fentanyl is as shown in formula (I):

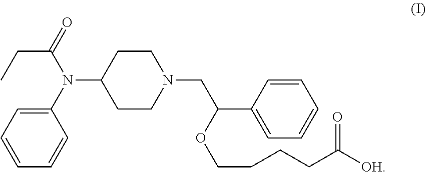 [SMILES: CCC(=O)N(c1ccccc1)C1CCN(CC(OCCCCC(=O)O)c2ccccc2)CC1]

2. The artificial hapten of fentanyl according to claim 1, wherein the artificial hapten of fentanyl is prepared by the following method, which comprises:
step (1): dissolving β-hydroxyfentanyl in N,N-dimethylformamide, adding sodium hydride, and stirring the mixture at room temperature for reaction; then adding ethyl 5-bromovalerate, and refluxing and stirring the mixture for reaction; and after ending the reaction, removing a solvent and extracting the mixture with ethyl acetate, and filtering, drying and purifying the mixture to obtain an oily substance A; and
step (2): dissolving the oily substance A with a mixed solution of tetrahydrofuran and anhydrous methanol, adding a sodium hydroxide aqueous solution, stirring the mixture at room temperature for reaction and then adjusting a pH value to be 4 to 5, extracting the mixture with dichloromethane for multiple times, combining organic phases, drying and filtering the mixture, removing dichloromethane and then purifying the mixture to obtain the artificial hapten of fentanyl.

3. The artificial hapten of fentanyl according to claim 2, wherein a molar ratio of the ε-hydroxyfentanyl to the sodium hydride in the step (1) is (1 to 1.5):3.

4. The artificial hapten of fentanyl according to claim 2, wherein a molar ratio of the β-hydroxyfentanyl to the ethyl 5-bromovalerate in the step (1) is 1:(1.5 to 2).

5. The artificial hapten of fentanyl according to claim 2, wherein an equivalent concentration of the sodium hydroxide aqueous solution in the step (2) is 1 N to 1.5 N.

6. The artificial hapten of fentanyl according to claim 2, wherein a mass-to-volume ratio of the oily substance A to the tetrahydrofuran, the anhydrous methanol and the sodium hydroxide aqueous solution in the step (2) is 100 mg:(1.5 to 2) mL:(1.8 to 2) mL:10 mL.

7. An artificial antigen of fentanyl, obtained by coupling the artificial hapten of fentanyl according to claim 1 with a carrier protein, wherein a molecular structural formula of the artificial antigen of fentanyl is as shown in formula (II): 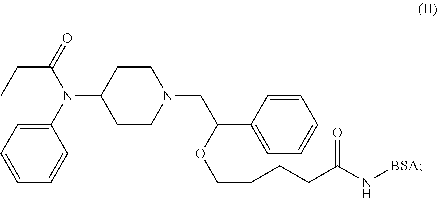
in formula (II), BSA refers to bovine serum albumin.

8. The artificial antigen of fentanyl according to claim 7, wherein the artificial antigen of fentanyl is prepared by the following method, which comprises:
step (1): dissolving the artificial hapten of fentanyl in a mixed solution of DMF and triethylamine for stirring in ice bath, adding isobutyl chloroformate during the stirring, and continuously stirring the mixture in ice bath for reaction; and after ending the reaction, centrifuging the mixture to obtain a supernatant; and
step (2): dropwise adding the supernatant obtained in the step (1) into a bovine serum albumin solution, standing the mixed solution obtained at 3° C. to 5° C. overnight, dialyzing and centrifuging the mixture, and taking a supernatant to obtain the artificial antigen of fentanyl.

9. The artificial antigen of fentanyl according to claim 8, wherein the mixture is continuously stirred in ice bath for reaction for 2 hours to 2.5 hours after adding the isobutyl chloroformate in the step (1).

10. The artificial antigen of fentanyl according to claim 8, wherein a concentration of the bovine serum albumin solution in the step (2) is 5 mg/mL.

11. The artificial antigen of fentanyl according to claim 8, wherein a volume ratio of the supernatant to the bovine serum albumin solution in the step (2) is 1:(5 to 6).

12. An application of an anti-fentanyl antibody in immunodetection of fentanyl in a sample, wherein the anti-fentanyl antibody is obtained by animal immunization with the artificial antigen of fentanyl according to claim 7.

13. The application according to claim 12, wherein the sample comprises a body fluid sample, a tissue sample or an environmental sample.

14. The application according to claim 13, wherein the body fluid sample refers to a mammal or human body fluid sample, which comprises urine, saliva, sweat or blood.

15. The application according to claim 13, wherein the tissue sample refers to a mammal or human tissue sample, which comprises hair, nail or liver.

16. The application according to claim 13, wherein the environmental sample comprises powder, an object surface residue or a water sample.

17. The application according to claim 12, wherein the immunodetection comprises a competitive inhibition ELISA assay, a competitive enzyme-linked immunosorbent assay or an immunochromatography assay.

18. The application according to claim 17, wherein the competitive inhibition ELISA assay specifically comprises using the anti-fentanyl antibody to prepare a competitive inhibition ELISA assay kit for fentanyl, and the competitive inhibition ELISA assay kit for fentanyl comprises the anti-fentanyl antibody, a fentanyl standard and an enzyme-labeled secondary antibody.

19. The application according to claim 17, wherein the competitive enzyme-linked immunosorbent assay specifically comprises using the anti-fentanyl antibody to prepare a competitive enzyme-linked immunosorbent assay kit, and the competitive enzyme-linked immunosorbent assay kit comprises an enzyme-labeled plate wrapped with the anti-fentanyl antibody, a fentanyl standard, an enzyme-labeled antigen, a substrate and a termination solution.

20. The application according to claim 17, wherein the immunochromatography assay specifically using the comprises anti-fentanyl antibody to prepare a fentanyl immunodetection test strip, and the fentanyl immunodetection test strip comprises a sample pad, a marker pad, a reaction membrane and a sample suction pad; and the marker pad is wrapped with the anti-fentanyl antibody, a detection line of the reaction membrane is wrapped with a fentanyl-bovine serum albumin complex, and a quality control line of the reaction membrane is wrapped with a secondary antibody.
